(19) United States
(12) Patent Application Publication
(10) Pub. No.: US 2009/0041859 A1
(43) Pub. Date: Feb. 12, 2009

(54) Wound Treatment Drugs for External Use

(75) Inventors: Hitoshi Mizutani, Mie (JP); Yoshiaki Miyamoto, Osaka (JP); Atsushi Nishikawa, Shiga (JP)

Correspondence Address: Warren M Cheek, Jr;Wenderoth, Lind & Ponack, L.L.P., 2033 K Street, N.W., Suite 800, Washington, DC, 20006, US

(73) Assignees: Mie University, Mie (JP); MARUISHI PHARMACEUTICAL CO., LTD., Osaka (JP)

(21) Appl. No.: 11/887,975

(22) PCT Filed: Apr. 7, 2006
(86) PCT No.: PCT/JP2006/307472

§ 371 Date: Jul. 2, 2008

(30) Foreign Application Priority Data
Apr. 8, 2005 (JP) 2005-112174

(51) Int. Cl. A61K 33/18; A61P 17/02

(52) U.S. Cl. 424/667

(57) ABSTRACT

[Problems] The objective is to provide a new low-toxic drug for external use for intractable diseases, such as erosion, decubitus, and skin ulcer, that prevents cicatrization and reduces iodine accumulation on wound surfaces with sufficiently promoting healing.

[Means for solving problem] Preparation of a wound treatment drug for external use containing oily bases and iodine but substantially no white soft sugar, which is a characteristic of the drug of this invention. The invented drug prevents infection through the wound surface due to a sufficient bactericidal effect of iodine, and promotes wound healing, prevents cicatrization and reduces iodine accumulation on the wound surface by maintenance of appropriate moisture in the wound surface due to a moisturizing effect of oily bases.

### CROSS-REFERENCE TO RELATED APPLICATIONS

This application claims the benefit of PCT application Ser. No. PCT/JP2006/307472, filed Apr. 7, 2006, published as WO 2006/109734 A1, which claims the benefit of Japanese Patent Application No. 2005-112174 filed on Apr. 8, 2005, the entire contents of each of which are incorporated herein by reference.

### BACKGROUND OF THE INVENTION

1. Field of the Invention

This invention is related to a drug for external use for treatment of diseases accompanied by skin injuries, such as erosion, decubitus, skin ulcer, and burns.

2. Description of the Background Art

Skin tissue and mucosa are often injured by various causes.

These skin tissue injuries and ulcer are generally called ‘wounds’. For example, erosion, cut, abrasion, burns, decubitus, and skin ulcer are included.

These wounds are painful, and may cause infection. Further, keloid-like lesions are caused and scars may remain when problems occur in the wound healing process. Thus, it is preferable that wounds are completely healed in the early stage.

Particularly, decubitus and skin ulcer are likely to occur in not only bed-ridden elderly persons but also persons with disabilities, the poorly nourished, and weak recovering abilities. Decubitus and skin ulcer have been a social problem because these persist for a prolonged period.

Decubitus represents a pathology in which skin compression inhibits local blood flow, resulting in injury of the skin tissue due to circulatory failure. Mild decubitus is a sore on the skin surface, but skin ulcers are formed in the subcutaneous tissue, bone, and ligament in severe decubitus.

Severe decubitus is very intractable, and it may lead to death due to bacterial infection.

Development of various drugs for wound treatment to promote wound healing, particularly those for external use, has recently been progressing.

Examples include fibrinolytic enzymes, such as fibrinolysin, deoxyribonuclease, streptokinase, and streptodornase, necrotomy tissue agents containing lysozyme chloride, antimicrobial agents containing gentamicin sulfate, sulfadiazine silver, bacitracin, and fradiomycin sulfate, incarnant agents containing trafermin, bucladesine sodium, tretinoin tocoferil (tocoretinate), alprostadil alfadex, solcoseryl (extract from hemolysed blood of young cattle), and alcloxa, iodine preparations containing white soft sugar, povidone iodine, and iodine, and preparations containing bendazac, dimethyl isopropylazulene (guaiazulene), and epinephrine as active ingredients.

Especially, white soft sugar is known to exhibit a wound healing effect, and povidone iodine has bactericidal activity (due to iodine). Wound treatment drugs for external use consisting of a mixture of white soft sugar and povidone iodine have been used for treatment of wounds, such as decubitus and skin ulcer, as preparations that exhibit both wound healing and bactericidal effects.

Currently, white soft sugar/povidone iodine combined drugs with improved pharmaceutical stability and homogeneity are commercially available and widely used for treatment of wounds including decubitus in clinical practice.

Various wound treatment drugs for external use including white soft sugar/povidone iodine-combined-drugs and various wound protective materials as medical devices are supplied for medical practice, and wound treatment means are progressing. However, decubitus and skin ulcer are still intractable diseases, and promotion of healing of decubitus will become an important issue as society ages.

Drugs for external use targeting promotion of wound healing generally contain white soft sugar at a high concentration to increase the effect (Publication of patent application No. 2000-38342). However, these drugs for external use do not promote wound healing. Further, these drugs for external use induce a negative reaction such as cicatrization.

Many of these previous drugs contain water-soluble bases, prioritizing drying and cleanliness of the wound surface, but protection of the wound surface is not considered.

Moreover, iodine preparations described above are accumulated on the wound surface and surrounding skin. Residual iodine is a concern of adverse events, such as skin staining (so called iodine burns) and irritation by iodine preparation themselves. These drugs have many other problems, such as contraindication for patients with specific diseases.

Accordingly, development of a new low-toxic drug for external use that prevents cicatrization and reduces iodine accumulation on the wound surface with sufficiently promoting healing has been strongly needed for treatment of intractable diseases, such as decubitus and skin ulcer.

The objective of this invention is to provide a new low-toxic drug for external use for intractable diseases, such as decubitus and skin ulcer, that prevents cicatrization and reduces iodine accumulation on the wound surface with sufficiently promoting healing, in view of the above problems.

### SUMMARY OF INVENTION

In one embodiment of the present invention, a wound treatment drug for external use comprising oily base and iodine, wherein the content of said oily base is 50-99 weight %, wherein said drug contains substantially no white soft sugar.

In another embodiment of the present invention, a wound treatment drug for external use comprising oily base and iodine, wherein the content of said oily base is 50-99 weight %, wherein said drug contains no white soft sugar.

Yet in another embodiment of the present invention, a wound treatment drug for external use, wherein said oily base is one or more materials selected from petrolatum and gelated hydrocarbon.

Yet in another embodiment of the present invention, a wound treatment drug for external use, wherein said petrolatum is one or more materials selected from the group consisting of white petrolatum and hydrophilic petrolatum.

Yet in another embodiment of the present invention, a wound treatment drug for external use, wherein the content of said iodine is 0.01-5 weight %.

Yet in another embodiment of the present invention, a wound treatment drug for external use, wherein the dosage form of said drug is selected from paste, ointment, cream, liquid, gel, adhesive, cataplasm, and patch.

Yet in another embodiment of the present invention, a wound treatment drug for external use, wherein said wound is one selected from the group consisting of erosion, cut, abrasion, burns, decubitus, and skin ulcer.

A wound treatment drugs for external use of this invention prevents infection through wound surface by the bactericidal action, and maintains appropriate moisture of the wound surface by preventing excessive drying of the wound surface.

Subsequently, the drug of this invention enhances the wound treatment effect, prevents cicatrization, and reduces iodine accumulation on the wound surface.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1-a is photographs of the wound surface taken over time in a rat in the control group.

FIG. 1-b is photographs showing the therapeutic effect of U-PASTA on the rat wound surface.

FIG. 1-c is photographs showing the therapeutic effect of a drug of this invention on the rat wound surface.

FIG. 2 is photographs of the hematoxylin-eosin-stained rat wound tissues showing the therapeutic effect of a drug of this invention.

FIG. 3 shows the therapeutic effect of a drug of this invention presented as the wound area in mice.

FIG. 4 is photographs of the hematoxylin-eosin-stained mouse wound tissues showing the therapeutic effect of a drug of this invention.

### DETAILED DESCRIPTIONS

As a result of a keen study, the inventors found that a wound treatment drug for external use composed of a combination of iodine with a bactericidal effect and oily bases with a moisturizing effect substantially containing no white soft sugar, which has a wound healing effect, is useful for treatment of intractable diseases such as decubitus and skin ulcer, and completed this invention.

A wound treatment drug for external use of this invention prevents infection through wound surfaces by sufficient bactericidal action of iodine, and at the same time, a moisturizing effect of oily bases enables maintenance of appropriate moisture of the wound surface, which result in promotion of healing, prevention of cicatrization, and reduction of residual iodine on the wound surface.

The inventors also discovered that the absence of white soft sugar, which is contained at a high concentration in the current technique to promote wound healing, prevents excessive drying of wound surfaces. They also discovered that the drug promotes healing without inducing cicatrization.

Thus, the substantial absence of white soft sugar is a characteristic of the wound treatment drug for external use of this invention. This characteristic avoids negative effects, such as reduction of the healing effect due to excessive drying of wound surfaces and induction of cicatrization.

The specific point of this invention is that the preparations contain oily bases, such as petrolatum and gelated hydrocarbon, enables to maintain appropriate moisture of wound surfaces. That results in promotion of healing, prevention of cicatrization, and reduction of residual iodine on the wound surface.

Iodine and its preparations have been used as disinfectants for a long time, and oily bases, such as petrolatum and gelated hydrocarbon, are widely used as skin protective agents and bases of various preparations, and their safety has been confirmed.

However, the above combination has not been applied as active ingredients of therapeutic drugs for diseases accompanied by skin injuries, such as erosion, decubitus, skin ulcer, and burns.

Embodiments for carrying out this invention to exert these effects are described in detail below.

Any oily base generally used for ointments can be used for this invention, but petrolatum and gelated hydrocarbon are preferably used.

Oily bases mixed with petrolatum and gelated hydrocarbon may also be used.

‘Petrolatum’ used for this invention may be white, yellow, and hydrophilic petrolatum. Preferably, ‘white petrolatum’ and ‘hydrophilic petrolatum’ are used.

‘White petrolatum’ is prepared by decolorization and purification of a mixture of petrolic hydrocarbons. And products listed in the Japanese Pharmacopoeia may be used as white petrolatum.

‘White petrolatum’, which is generally available, includes ‘ointment base Japanese Pharmacopoeia white petrolatum 500 g’ of Maruishi Pharmaceutical Co., Ltd.

‘Hydrophilic petrolatum’ is a mixture of white petrolatum, white beeswax, stearyl alcohol, and cholesterol. And products listed in Japanese Pharmacopoeia may be used as hydrophilic petrolatum.

‘Hydrophilic petrolatum’, which is generally available, includes ‘ointment base Japanese Pharmacopoeia hydrophilic Petrolatum 500 g’ of Maruishi Pharmaceutical Co., Ltd.

‘Gelated hydrocarbon’ used for this invention includes a mixture of liquid paraffin and polyethylene resin. ‘Gelated hydrocarbon’, which is generally available, includes ‘Plastibase’ (registered trademark) of Taisho Pharmaceutical Co., Ltd.

The content of oily bases, such as petrolatum and gelated hydrocarbon, in a wound treatment drug for external use of this invention is 50-99 weight %, preferably, 70-97 weight %, and more preferably, 85-95 weight %.

The moisturizing effect is not sufficiently exerted when the content is less than 50 weight %, and preparation is difficult when the content is higher than 99 weight %. Thus, these contents are inappropriate.

‘Iodine’ used in this invention is not specified, and it may be used as iodophors, such as complexes with 1-vinyl-2-pyrrolidone polymer (povidone iodine) and surfactant.

The content of ‘iodine’ in a wound treatment drug for external use is 0.01-5 weight %, preferably 0.05-2 weight %, and more preferably 0.1-0.9 weight %.

The bactericidal effect is not sufficiently exerted when the content is less than 0.01 weight %. Iodine preparations accumulate on the wound surface and surrounding skin when the content is higher than 5 weight %. And the residual iodine may exhibit adverse effects, such as skin staining (so called iodine burns) and irritation by iodine preparations themselves. Thus, these contents are inappropriate.

As a characteristic of a wound treatment drug for external use of this invention, the drug contains substantially no white soft sugar, which is considered to have wound healing action.

Such compositions avoid negative effects: reduction in the healing effect due to excessive drying of the wound surface and induction of cicatrization.

White soft sugar described here includes hygroscopic sugar group, such as purified sugar, for example, Japanese Pharmacopoeia listed white soft sugar and sucrose.

The sugar group induces cicatrization of the wound surface.

Current wound treatment drugs for external use contain white soft sugar at a high concentration (50-90 weight %), and the wound healing effect of white soft sugar has been considered to promote wound healing. However, these drugs have not sufficiently exhibited the expected effect, but induced cicatrization.

The inventors discovered that the above negative effects due to excessive drying of wound surfaces can be prevented by containing substantially no sucrose at a high concentration, and subsequently developed a wound treatment drug for external use of this invention characterized in containing substantially no white soft sugar.

‘Containing substantially no white soft sugar’ means containing no white soft sugar or containing white soft sugar at a level which does not exert pharmacological activities, such as wound healing.

The level which does not exert pharmacological activities may be any content lower than the minimum combined amount required for exertion of pharmacological activities of white soft sugar, but less than 20 weight % is preferred.

A wound treatment drug for external use of this invention contains an oily base and iodine preparation described above as essential ingredients, and substantially no white soft sugar. The drug prevents infection through wound surfaces due to a sufficient bactericidal effect of iodine therein, and enables to maintain appropriate moisture of wound surfaces due to moisturizing effects of oily bases therein. As a result, the wound treatment effect may be enhanced.

Furthermore, the drug enhances wound healing, prevents cicatrization, and reduces iodine accumulation on wound surfaces by maintaining moist wound surfaces.

At the same time, the drug does not exhibit negative effects, such as delay in healing and induction of cicatrization, because of containing substantially no white soft sugar which may cause excessive drying of wound surfaces.

A wound treatment drug of this invention can be formulated in known dosage forms for external use using the above active ingredients and pharmacologically acceptable drug carriers.

Dosage forms are preferably paste, ointment, cream, liquid, gel, adhesive, cataplasm, and patch.

A wound treatment drug for external use of this invention can be prepared and manufactured corresponding to the above dosage forms in which the above iodine components are mixed and homogenized by the standard method using conventional pharmaceutical techniques.

For example, the drug is prepared by slowly adding various bases and specific components to povidone iodine and white petrolatum, kneading them and homogenizing them.

In addition to the above active ingredients, components below are included as examples of base components used in the drug of this invention.

As for lipid, for example, middle-chain triglyceride, synthetic oil, such as hard fat, plant oils, such as olive oil, soy bean oil, canola oil, peanut oil, safflower oil, rice bran oil, sesame oil, camellia oil, corn oil, cotton seed oil, and coconut oil, animal oils, such as lard and beef tallow, and solidified oils of these are used.

As for wax, for example, natural waxes, such as lanolin, yellow beeswax, carnauba wax, and spermaceti, and mineral waxes, such as montan wax, and synthetic wax, are used.

As for hydrocarbon, for example, paraffin, liquid paraffin, microcrystalline wax, squalene, polyethylene powder, and gelated hydrocarbon are used.

As for higher fatty acid, for example, stearic acid, behenic acid, palmitic acid, and oleic acid are used.

As for higher alcohol, for example, cetanol, stearyl alcohol, oleyl alcohol, and cholesterol are used. For polyhydric alcohol, for example, propylene glycol, polyethylene glycol, glycerin, and 1,3-butylene glycol are used.

As for synthetic and natural macromolecules, for example, carrageena, starch, dextrin, dextrin polymer (cadexomer), polyoxyethylene polyoxypropylene glycol (poloxamer), tragacanth, Arabic gum, locust bean gum, pectin, gelatin, xanthan gum, pullulan, alginate, hydroxypropylcellulose, sodium carboxymethylcellulose, polyacrylate, polyvinyl alcohol, polyvinyl pyrrolidone, and carboxyvinyl polymer are used.

As for surfactant, for example, alkyl sulfate, polyoxyethylene alkylether phosphate, glycerin fatty acid ester, polyglycerin fatty acid ester, sorbitan fatty acid ester, polyoxyethylene sorbitan fatty acid ester, polyxyethylene solidified castor oil, polyethylene glycol fatty acid ester, polyoxyethylene polyoxypropylene glycol, polyoxyethylene alkyl ether, and sucrose fatty acid ester are used.

As for lower alcohol, for example, ethanol and isopropyl alcohol are used.

As for ketone, for example, acetone and methylethyl ketone are used.

As for powder, for example, kaolin, talc, zinc oxide, titanium oxide, magnesium stearate, anhydrous silicic acid, and starch are used.

As for cellulose derivative, for example, methylcellulose, sodium carboxymethylcellulose, and hydroxypropylcellulose are used.

As for inorganic salt, for example, potassium iodide, sodium iodide, sodium sulfate, sodium hydrogen carbonate, sodium chloride, calcium nitrate, potassium nitrate, sodium nitrate, aluminum sulfate, sodium polyphosphate, ammonium chloride, iron sulfate, sodium phosphate, magnesium sulfate, sodium thiosulfate, sodium sesquicarbonate, sodium sulfate, borax, calcium oxide, magnesium carbonate, and potassium chloride are used.

The relationship between each dosage form and additives are more concretely described below.

As for paste, the drug can be used as oily paste. As for the base of-paste, for example, lipids, waxes, and hydrocarbons are used.

As for ointment, for example, lipids, polyhydric alcohols, and hydrocarbons are used as the base.

As for cream, for example, surfactants, higher alcohols, higher fatty acids, hydrocarbons, polyhydric alcohols, and water (purified water) are used as the base.

As for liquid and gel, for example, water (purified water), lower alcohols, ketones, lipids, polyhydric alcohols, surfactants, hydrocarbons, and synthetic and natural macromolecules are used as the base.

In addition, alkali metal hydroxides, such as sodium hydroxide, may be used as a pH adjuster, as needed in the drug of this invention. Antiseptics/preservatives may also be combined, for example, alkali metal benzoates, such as sodium benzoate, p-hydroxybenzoate esters, and sorbic acid.

Furthermore, antioxidants, for example, tocopherol, dibutyl hydroxytoluene, and butyl hydroxyanisole, may be used, as needed.

The wound treatment drug for external use of this invention prepared above may also be formulated as emulsion, for which w/o oily types can be used.

The drug for skin application of this invention may be directly applied to affected regions. Furthermore, for example, adhesives, such as cataplasms and plasters prepared by spreading the drug on elastic and nonwoven and plastic sheets, may be applied to affected regions.

The wound treatment drug for external use of this invention prepared above is safe for skin and results in comfortable use. They are also clinically superior and extremely effective for promoting wound healing.

Thus, the drug exhibits a sufficient effect on healing of wounds: erosion, cut, abrasion, burns, decubitus, and skin ulcer.

### EXAMPLES

The invention is explained with examples below, but not limited to the examples.

Combined amounts are presented as weight %, not otherwise specified.

### [Example 1 ] Effect On The Wound Healing Process In Rats

A wound treatment drug for external use of this invention for skin application (ointments) was prepared using the following formula and method.

Drugs used for the formula example are shown in 1) and 2).

1) Hydrophilic petrolatum listed in Japanese Pharmacopoeia (Maruishi Pharmaceutical Co., Ltd.)

The preparation contained 8 g of white beeswax, 3 g of stearyl alcohol, 3 g of cholesterol, an appropriate amount of white petrolatum, and dibutyl hydroxytoluene as an additive per 100 g of the above hydrophilic petrolatum. This preparation is used as an ointment base and skin protector.

2) Povidone iodine (Sigma Co.)

3) Polyethylene glycol 400 (Wako Pure Chemical Industries Ltd.)

### (Formula 1 (100 g))

Hydrophilic petrolatum: 90.0 g

Povidone iodine: 3.0 g

Polyethylene glycol 400:3.5 g

Purified water: 3.5 g

### (Control Drug)

Formula of U-PASTA KOWA (100 g)

Sucrose: 70.0 g

Povidone iodine: 3.0 g

Other additives, such as macrogol

### (Production Method)

The substances were mixed at the above ratio and kneaded to a semi-solid state.

### (Promotion Of Wound Healing In Rats)

### [Experimental Methods]

S1c:SD rats (male, 6 weeks of age) were used. Under pentobarbital anesthesia, the rats were restrained on a recumbent position. The dorsal skin was slightly stretched and punched using a punch for leather (diameter: 1.8 cm). Symmetric full-thickness defective wounds were made by this procedure. Formula 1 and a commercial drug for treatment of decubitus containing 3% povidone iodine and 70% white soft sugar, U-PASTA KOWA (Kowa Co., Ltd.), were individually applied once a day at 150 mg to the wounds for 7 days.

In a control group, wounds were made and mock treatment was applied to the wounds for 7 days.

The dorsal region was photographed using a digital camera 3 times a week, and the wound areas were measured using imaging analysis software, NIH Image (the National Institutes of Health of the U.S.A.).

The wound area regards the mean of the right and left wound areas of each rat, and the mean and standard deviation of 5 rats were presented.

The dorsal skin including the wound surfaces was excised after application for 7 days in each group, and fixed in 10% neutral buffered formalin solution. Paraffin sections were prepared, stained with hematoxylin-eosin, and observed including pathological observation.

Based on Table 1<Reduction of wound area and healing rate>shown below, the wound area was reduced, and the healing rate was about 64% in the Formula 1 treatment group. In contrast, no significant differences were noted between the U-PASTA treatment and untreated control groups.

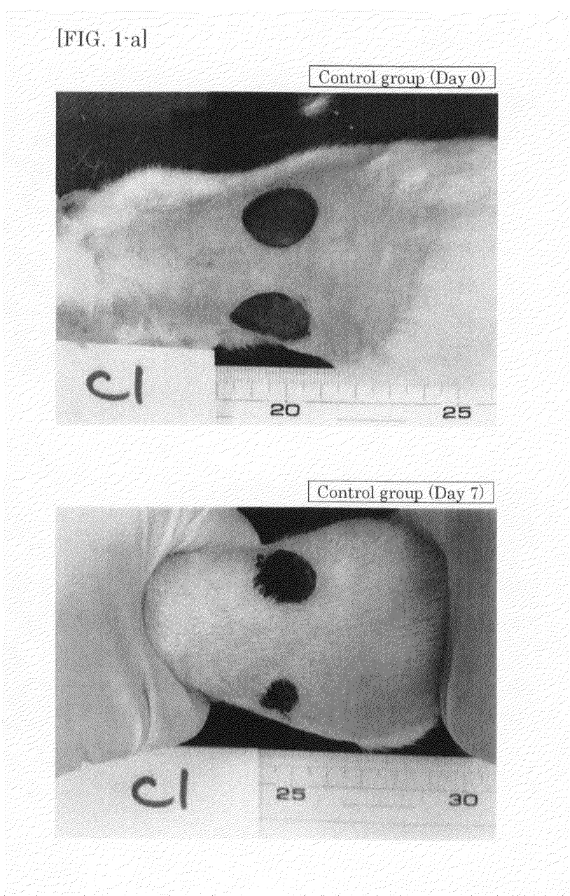

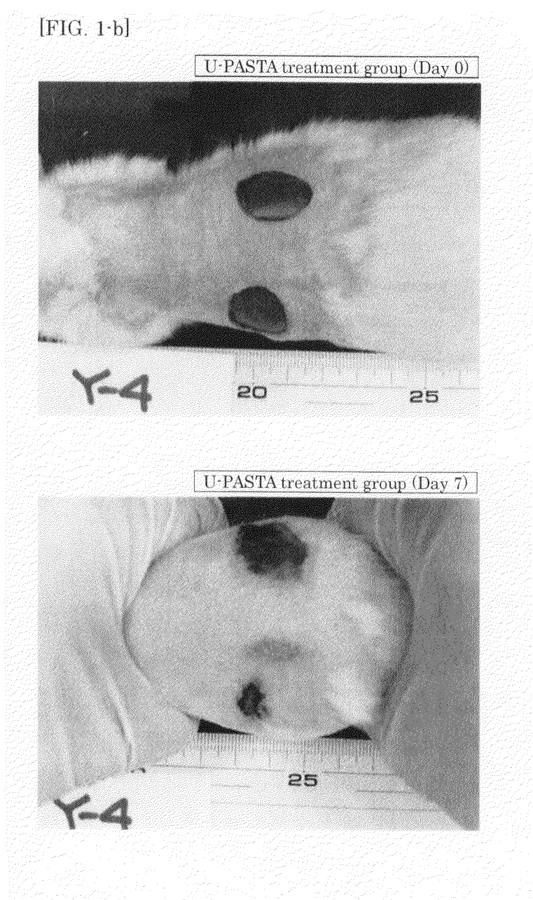

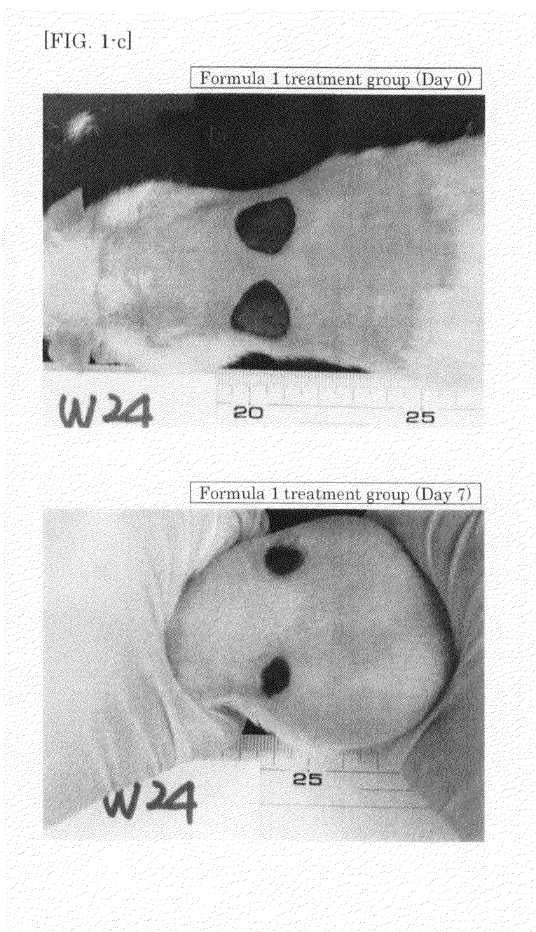

The appearance of the wound healing process is shown in FIG. 1.

In the control (FIG. 1-a) and U-PASTA treatment (FIG. 1-b) groups, very thick scab were formed. Scars on the wound surface and brown dirt showing residual iodine around the wound were noted on one side in the U-PASTA treatment group.

In the Formula 1 treatment group (FIG. 1-c), no thick scab or brown dirt around the wound were noted, showing ‘clean’ healing of wound surface.

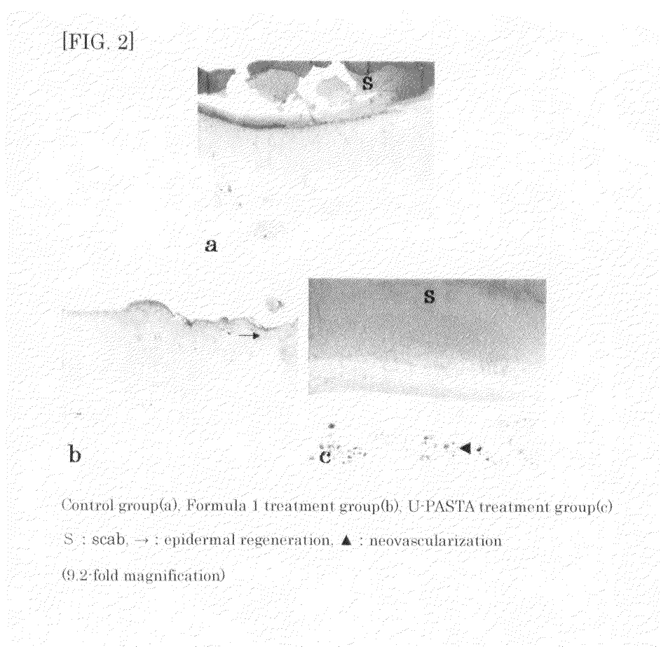

The results of hematoxylin-eosin staining of the wounded skin tissues are shown in FIG. 2.

Granulation was noted in the wounds in all rats in all groups. Especially, in the Formula 1 treatment group, epidermal regeneration was noted in the wound margin (FIG. 2-b).

In a few rats of the U-PASTA treatment group, fibrin aggregates and dead cells remains in the features of the early granulation period, and the features and marked subcutaneous neovascularization were partially noted. (FIG. 2-c).

White soft sugar contained in U-PASTA may have absorbed exudate containing cytokines and growth factors, which reduced fibroblast migration and proliferation, and delayed granulation.

TABLE 1

| | Wound area (cm²) | | Healing rate |
|---|---|---|---|
| Experiment group | Day 0 | Day 7 | (%) |
| Control group (untreated) | 2.49 ± 0.33 | 1.23 ± 0.22 | 49.5 ± 14.9 |
| Formula 1 treatment group | 2.17 ± 0.10 | 0.79 ± 0.18 | 63.6 ± 7.5 |
| U-PASTA treatment group | 2.00 ± 0.29 | 0.97 ± 0.27 | 52.1 ± 8.4 |

The wound area was measured in 5 rats in each group, and presented as the mean±standard deviation.

The healing rate was calculated by the equation below (Equation 1).

### [Equation 1]

Healing rate (%)=100×{1-(wound area after application for 7 days/wound area on day 0 of application)}

### [Example 2] Effect On The Wound Healing Process In Mice

A wound treatment drug for external use of this invention for skin application (ointments) was prepared using the following formula and method.

### [Formula Example]

Drugs used for the formula example are shown below.

White petrolatum (Nacalai Tesque Inc.)

Isodine gel (Meiji Seika Kaisha Ltd.)

A commercial disinfectant for disinfection of wound and skin burn surfaces containing 100 mg of povidone iodine and macrogols 4000, macrogols 6000, and macrogols 400 per 1 g.

### (Formula 2 (100 g))

White petrolatum: 90.0 g

Isodine gel: 10.0 g

### (Production Method)

Isodine gel and white petrolatum were mixed at the above ratio and kneaded to a semi-solid state.

### <Promotion Of Wound Healing In Mice>

### [Methods]

S1c:ICR mice (male, 6 weeks of age) were used. Under pentobarbital anesthesia, the dorsal region was shaved, and the skin was punched using a disposable biopsy punch (diameter: 5 mm, Kai Industries Co., Ltd.) to make full-thickness defective wounds.

An appropriate amount of Formula 2 was applied for 7 days.

In a control group, wounds were made and mock treatment was applied to the wounds for 7 days.

The dorsal region was photographed using a digital camera 3 times a week, and the wound areas were measured using imaging analysis software, NIH Image (the National Institutes of Health of the U.S.A.).

The wound area was presented as the mean and standard deviation of 5 mices.

The dorsal skin including the wound surfaces was excised after application for 8 days in each group, and fixed in 10% neutral buffered formalin solution. Paraffin sections were prepared, stained with hematoxylin-eosion, and observed including pathological observation.

### [Results]

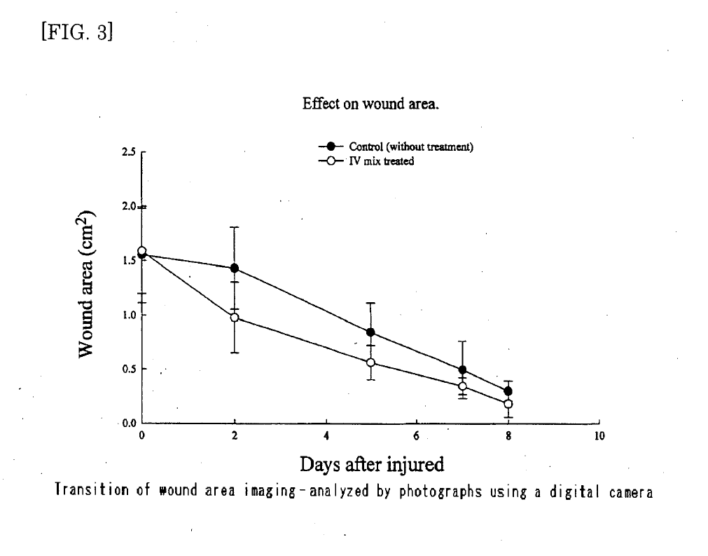

Based on Table 2<Reduction of wound area and the healing rate>shown below and FIG. 3, the wound area was reduced in both the control and Formula 2 treatment groups, but the healing rate in the Formula 2 treatment group was about 88% , showing faster healing than the control group.

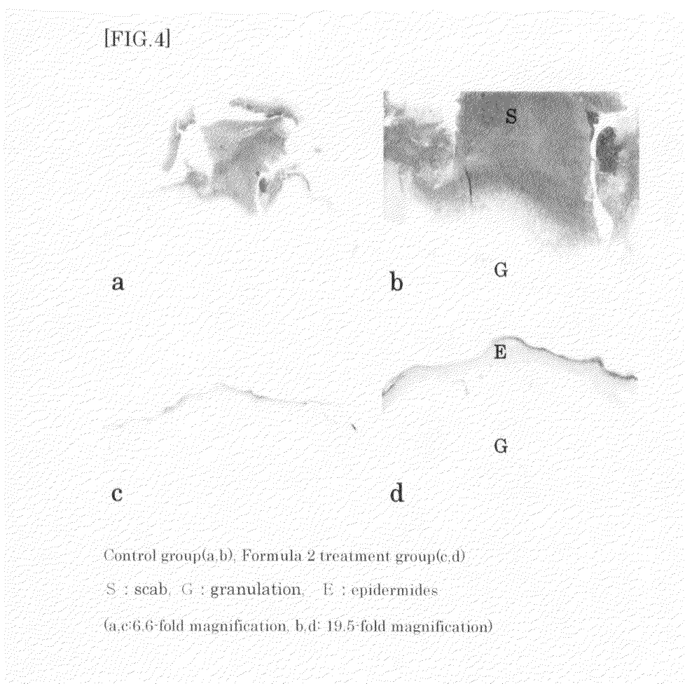

As shown in FIG. 4, granulation was noted in all mices in both the control and Formula 2 treatment groups, but new inflammatory reaction was not noted, suggesting that the injured tissue was in the repair (healing) step.

Epidermal regeneration was noted in all mices in the Formula 2 group, but only 2 of the 5 mices in the control group.

TABLE 2

| | Wound area (cm²) | | Healing rate |
|---|---|---|---|
| Experiment group | Day 0 | Day 7 | (%) |
| Control group (untreated) | 1.55 ± 0.44 | 0.30 ± 0.09 | 79.1 ± 9.5 |
| Formula 2 treatment group | 1.59 ± 0.39 | 0.18 ± 0.12 | 88.4 ± 7.4 |

The wound area was measured in 5 mices in each group, and presented as the mean±standard deviation.

The healing rate was calculated by the equation below (Equation 2).

### [Equation 2]

Healing rate (%)=100×{1-(wound area after application for 7 days/wound area on day 0 of application)}

The above examples 1 and 2 revealed the superior therapeutic effect on wounds of the wound treatment drug of this invention.

Another formula example of the drug for external use of this invention is shown below.

### (Formula 3 (100 g))

Hydrophilic petrolatum: 70.0 g

Povidone iodine: 3.0 g

Polyethylene glycol 400: 13.5 g

Purified water: 13.5 g

A practical formula example of the drug of this invention is shown below.

### (Formula 4 (100 g))

Iodine: 0.9 g

Sodium iodide: 1.7 g

Purified water: 3.0 g

Poloxamer 235: 4.0 g

Poloxamer 403: 1.0 g

Hydrophilic petrolatum: 89.4 g

1. A wound treatment drug for external use comprising oily base and iodine, wherein the content of said oily base is 50-99 weight %, wherein said drug contains substantially no white soft sugar.

2. A wound treatment drug for external use comprising oily base and iodine, wherein the content of said oily base is 50-99 weight %, wherein said drug contains no white soft sugar.

3. A wound treatment drug for external use according to claim 1, wherein said oily base is one or more materials selected from petrolatum and gelated hydrocarbon.

4. A wound treatment drug for external use according to claim 1, wherein said petrolatum is one or more materials selected from the group consisting of white petrolatum and hydrophilic petrolatum.

5. (canceled)

6. A wound treatment drug for external use according to claim 1, wherein the content of said iodine is 0.01-5 weight %.

7. A wound treatment drug for external use according to claim 1, wherein the dosage form of said drug is selected from paste, ointment, cream, liquid, gel, adhesive, cataplasm, and patch.

8. A wound treatment drug for external use according to claim 1, wherein said wound is one selected from the group consisting of erosion, cut, abrasion, burns, decubitus, and skin ulcer.
